(19) United States
(12) Patent Application Publication
(10) Pub. No.: US 2014/0358004 A1
(43) Pub. Date: Dec. 4, 2014

(54) SIMULTANEOUS ULTRASONIC VIEWING OF 3D VOLUME FROM MULTIPLE DIRECTIONS

(71) Applicants: David Prater, Andover, MA (US); Stephen Watkins, Windham, NH (US); David Frank Adams, Bradford, MA (US)

(72) Inventors: David Prater, Andover, MA (US); Stephen Watkins, Windham, NH (US); David Frank Adams, Bradford, MA (US)

(73) Assignee: KONINKLIJKE PHILIPS N.V., EINDHOVEN (NL)

(21) Appl. No.: 14/372,029

(22) PCT Filed: Feb. 11, 2013
(86) PCT No.: PCT/IB2013/051118

§ 371 Date: Jul. 14, 2014

Related U.S. Application Data
(60) Provisional application No. 61/597,931, filed on Feb. 13, 2012.

(51) Int. Cl. A61B 8/08; A61B 8/00

(52) U.S. Cl. CPC A61B 8/483; A61B 8/488; A61B 8/463
USPC 600/443

(57) ABSTRACT

An ultrasonic diagnostic imaging system scans a volumetric region of a body. A clinician defines a three dimensional region of interest within the volumetric region. The three dimensional region of interest is viewed from two different viewing directions to give the clinician a sense of the structure, makeup, and orientation of the region of interest. The three dimensional region of interest can be viewed from viewing directions in 180° opposition to each other, orthogonal, or at an intermediate angle. Manipulation of one view of the three dimensional region of interest causes both views to change, as if the clinician were manipulating both views simultaneously in the same way.

This invention relates to medical diagnostic ultrasound systems and, in particular, to ultrasonic imaging systems which display a 3D volume in simultaneous views from multiple directions.

Ultrasonic diagnostic imaging system have traditionally been used to image a plane of the body in real time. A probe with a one dimensional (1D) array transducer or mechanically swept single element transducer can be operated to repeatedly scan a plane of the body to produce real time image sequences for live display of the anatomy. Recently two dimensional (2D) array transducers and mechanically swept 1D arrays have been developed for scanning a volumetric region of the body. Such probes can be used to produce three dimensional (3D) images of the volume being scanning, also in real time. A display technique commonly used for 3D display of ultrasonically scanned volumes is called kinetic parallax, in which a 3D data set of the volume is rendered from a series of different viewing directions. As the operator moves a control on the ultrasound system to change the viewing direction, the volume rendering processor renders the volume in a newly selected viewing direction and the progression of different directions gives the appearance of a 3D volume moving on the display screen. Individual planes can be selected from a three dimensional data set for viewing, a technique known as multiplanar reconstruction (MPR).

It is at times desirable to view a volumetric region of interest (ROI) from different directions. With a conventional viewer this must be done by viewing the ROI from one direction, then turning or rotating the 3D ROI so that it can be seen from the second direction. A comparison of the two views must be done by remembering what was seen in the first view, then moving the view to the second direction and making the comparison based on the recollection of the first view. For comparison of subtle anatomical differences, it would be preferable not to rely on memorization, or moving the views back and forth to try to make the diagnosis. It would be preferable to be able to see both views simultaneously so that the clinician is seeing both views at the same time while making the diagnosis.

In accordance with the principles of the present invention, a diagnostic ultrasound system is described which enables a clinician to view a volume from multiple external viewing perspectives at the same time. When the clinician manipulates one view, the manipulation is applied to the second view so that both views are changed in unison, as the clinician would expect the views to change if both were altered in the same way. Either or both views can also be interrogated by MPR viewing. A system of the present invention is particularly useful for guiding an invasive device such as a needle or a catheter inside the body.

In the drawings:

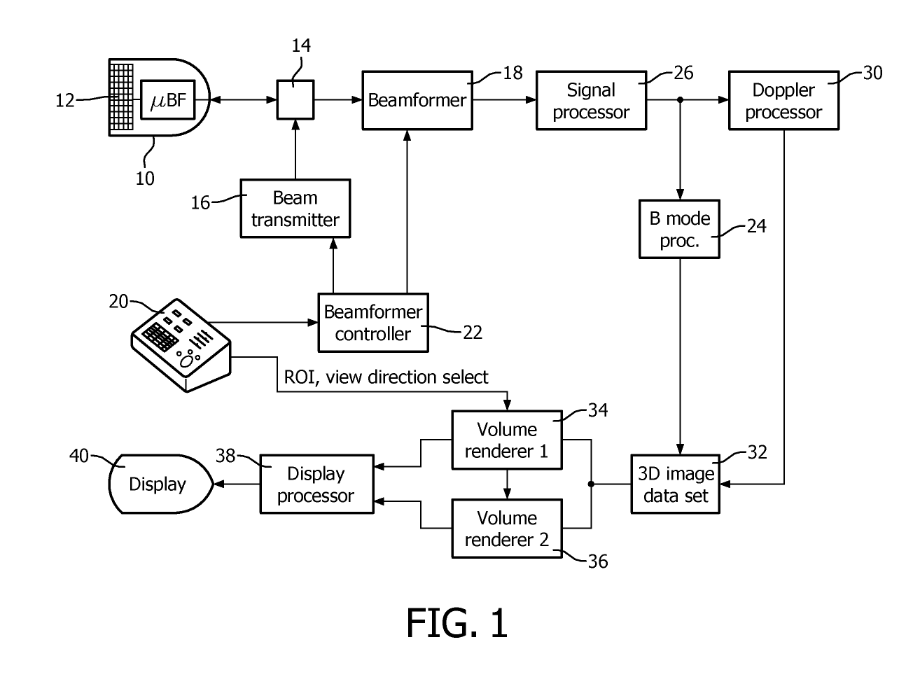

FIG. 1 illustrates in block diagram form an ultrasonic diagnostic imaging system constructed in accordance with the principles of the present invention.

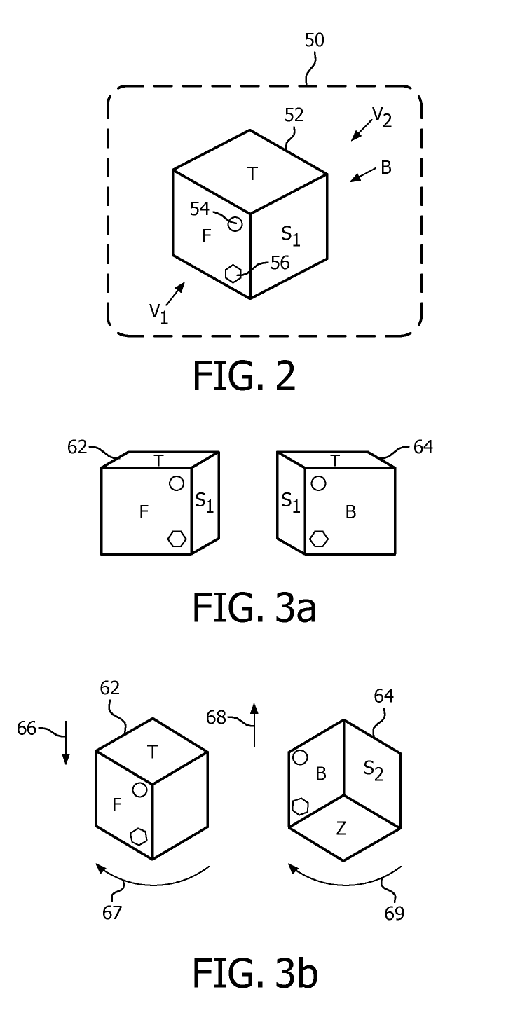

FIG. 2 shows a cubic ROI and two different viewing orientations.

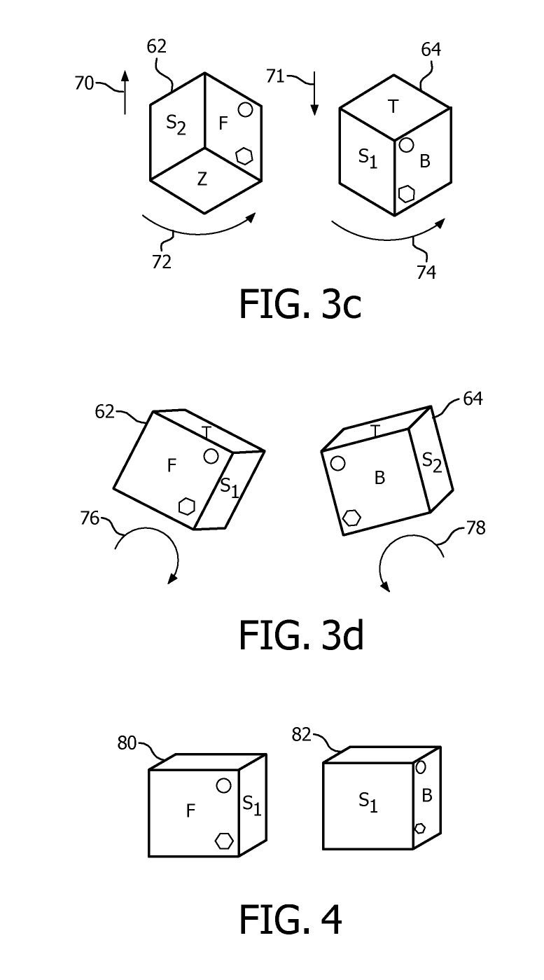

FIGS. 3a-3d illustrate simultaneous changes of two viewing orientations of the cubic ROI of FIG. 2 by manipulation of one of the views.

FIG. 4 illustrates two simultaneous views of the cubic ROI of FIG. 2 from orthogonal viewing orientations.

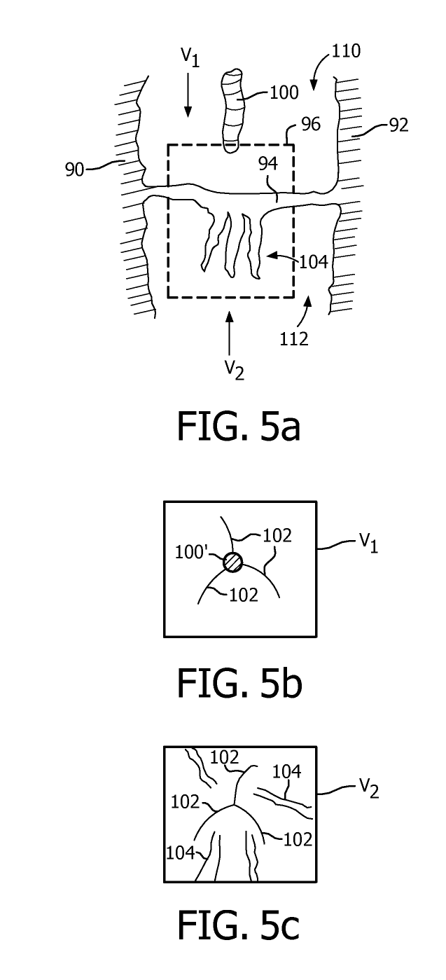

FIGS. 5a-5c illustrate simultaneous views from different directions of a volumetric ROI including a heart valve.

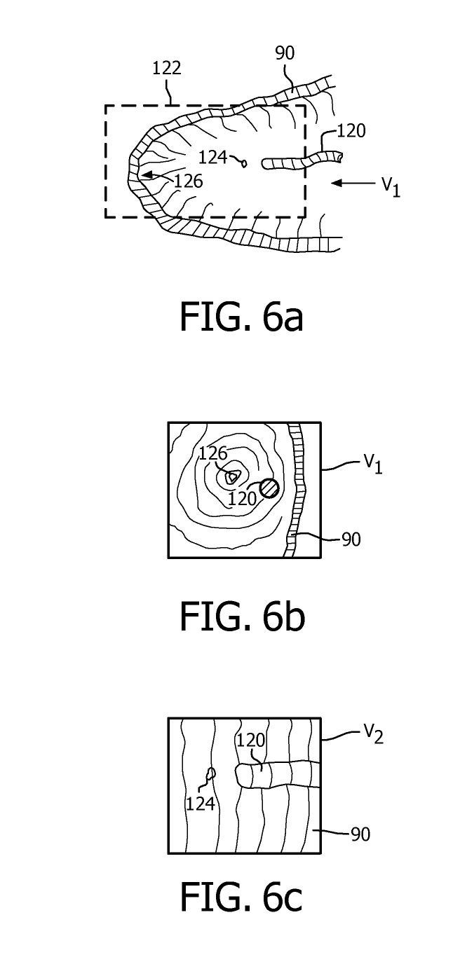

FIGS. 6a-6c illustrate simultaneous views of a catheter procedure from orthogonal viewing directions.

Referring first to FIG. 1, an ultrasonic diagnostic imaging system constructed in accordance with the principles of the present invention is shown in block diagram form. An ultrasound probe 10 capable of three dimensional imaging includes a two dimensional array transducer 12 which transmits electronically steered and focused beams over a volumetric region and receives single or multiple receive beams in response to each transmit beam. Groups of adjacent transducer elements referred to as “patches” or “subarrays” are integrally operated by a microbeamformer (μBF) in the probe 12, which performs partial beamforming of received echo signals and thereby reduces the number of conductors in the cable between the probe and the main system. Suitable two dimensional arrays are described in U.S. Pat. No. 6,419,633 (Robinson et al.) and in U.S. Pat. No. 6,368,281 (Solomon et al.) Microbeamformers are described in U.S. Pat. No. 5,997,479 (Savord et al.) and U.S. Pat. No. 6,013,032 (Savord). The transmit beam characteristics of the array are controlled by a beam transmitter 16, which causes the apodized aperture elements of the array to emit a focused beam of the desired breadth in a desired direction through a volumetric region of the body. Transmit pulses are coupled from the beam transmitter 16 to the elements of the array by means of a transmit/receive switch 14. The echo signals received by the array elements and microbeamformer in response to a transmit beam are coupled to a system beamformer 18, where the partially beamformed echo signals from the microbeamformer are processed to form fully beamformed single or multiple receive beams in response to a transmit beam. A suitable beamformer for this purpose is described in the aforementioned Savord '032 patent.

The receive beams formed by the beamformer 18 are coupled to a signal processor 26 which performs functions such as filtering and quadrature demodulation. The echo signals of the processed receive beams are coupled to a Doppler processor 30 and/or a B mode processor 24. The Doppler processor 30 processes the echo information into Doppler power or velocity information signals. For B mode imaging the receive beam echoes are envelope detected and the signals logarithmically compressed to a suitable dynamic range by the B mode processor 24. The echo and Doppler signals from the scanned volumetric region are processed to form one or more 3D image datasets which are stored in a 3D image dataset buffer 32. The 3D image data may be processed for display in several ways. One way is to produce multiple 2D planes of the volume. This is described in U.S. Pat. No. 6,443,896 (Detmer). Such planar images of a volumetric region are produced by a multi-planar reformatting as is known in the art. In accordance with the present invention, the three dimensional image data may also be rendered to form perspective or kinetic parallax 3D displays by volume renderers 34 and 36. The resulting images, which may be B mode, Doppler or both as described in U.S. Pat. No. 5,720,291 (Schwartz), are coupled to a display processor 38, from which they are displayed on an image display 40. User control of the beamformer controller 22, the selection of an ROI, the selection of directions in which the ROI is to be viewed, and other functions of the ultrasound system are provided through a user interface or control panel 20.

A clear understanding of manipulation of simultaneous views of a 3D ROI may be had with reference to FIGS. 2-4. In these drawings a cubic ROI 52 located in a volumetric region 50 is used for clarity of illustration. As seen in FIG. 2, the cubic ROI 52 has a front face F, a top face T, side faces S₁ and S₂, and back (B) and bottom (Z) faces, the latter three not visible in FIG. 2. The 3D ROI 52 has two passageways extending from the front face to the back face, one drawn as a circular passageway 54 and the other drawn as a hexagonal passageway 56. Two viewing directions V₁ and V₂ are also shown in FIG. 2, which view the 3D ROI from the front F and the back B, respectively.

FIGS. 3a-4 show simultaneous 3D views of the 3D ROI formed by simultaneous operation of volume renderer1 and volume renderer2 in accordance with the principles of the present invention. The two 3D views are displayed to the clinician simultaneously on the display 40 as illustrated in these drawings. Volume renderer1 renders the 3D ROI as viewed looking toward the front face F and volume renderer2 renders the 3D ROI as viewed looking toward the back face B. The viewing directions used for rendering are thus opposed to each other by 180°. In the front face view 62 of FIG. 3a the viewing direction is slightly to the right of and above the front face of the 3D ROI so that the top T and side S₁ faces can be seen. For the back face view 64 the viewing direction is slightly to the left of and above the back face B so that the side S₁ and top T faces can also be seen in this view. Slight variation from exactly 180° views can be used as shown in FIG. 3a, or both views can be exactly 180° in opposition as shown in FIG. 4. As FIG. 3a illustrates, the passageways 54,56 extending through the 3D ROI are seen on the right side of the front face F and on the left side of the back face B as a clinician would expect to see them.

In FIG. 3b the clinician has manipulated a control of the user interface such as a trackball on the control panel 20 or a softkey control on the display screen to rotate the 3D ROI 62 on the left side of the display slightly to the left as indicated by arrow 67. The clinician has also manipulated a user control to tilt the 3D ROI slightly downward as indicated by arrow 66 so that more of the top face T can be seen. As the clinician manipulates the left 3D ROI 62 in this way, the 3D ROI view 64 on the right moves in correspondence, as if the clinician manipulated the right view to move in the same way. The right view 64 from the back of the 3D ROI rotates the same amount to the left as indicated by the arrow 69 and tilts upward by the same amount (arrow 68) as the tilt of the left 3D ROI view, causing more of the bottom face Z to be visible. Thus, by manipulating one view of the 3D ROI, the corresponding adjustments are made to the other view of the 3D ROI. The clinician has the sense of moving one 3D ROI with the control adjustments and seeing the resulting change in both views of the front and back of the 3D ROI as if clinician were seeing the same ROI and its motion from two different views.

FIG. 3c illustrates the front and back 3D ROI views 62 and 64 after the clinician has rotated the ROI to the right (as indicated by arrows 72 and 74) and tilted the front view of the ROI up (as indicated by arrows 70) so that the bottom face Z is visible. As the drawing indicates, the back view 64 moves in a corresponding manner. The upward tilt 70 of the ROI as seen from the front is seen as a downward tilt from the back as indicated by arrow 71, causing the top face T to be more visible from the back. Both the left and right views move in unison as the clinician adjusts the orientation of one of the views.

FIG. 3d illustrates the result of rotating the left view to tilt the right side of the 3D ROI 62 downward. As this happens, the rear view 64 of the 3D ROI tilts down on the left side as indicated by arrow 78. This is how the clinician would expect the right view to behave when rotating the left view: the S₁ face side tilts down in both views. The same result can be obtained by tilting the right view 64 downward on the left side, which causes the corresponding effect of tilting the right side of view 62 down to the right. Thus, moving the ROI in one of the views causes the same movement of the other view, which is seen from the different viewing orientation.

FIG. 4 shows two views of a 3D ROI, with the left view 80 looking at the 3D ROI from the front face F and the right view 82 looking at the 3D ROI from the side face S. As in the previous examples, manipulating one of the views of the 3D ROI will cause the same motion of the 3D ROI in the other view but as seen from a different viewpoint. The two views of the 3D ROI can thus be at a 180° angle to each other as shown in FIGS. 3a-3d, or at a 90° angle to each other as shown in FIG. 4, or at any other intermediate angle between the views, e.g., between 0° and 180°.

FIGS. 5a-5c illustrate a clinical application of an ultrasound system of the present invention. In this example a catheter 100 has been threaded into an atrium 110 of a heart in preparation for passage through a mitral or tricuspid valve 94 and into a ventricle 112. The heart valve 94 is seen to be attached to the myocardial walls 90 and 92 on opposite sides of the heart. Extending from the valve leaflets in the ventricle are chordate tendineae 104, cord-like tendons that attach the valve leaflets to papillary muscles in the ventricle. An ultrasound system of the present invention is used to guide the catheter procedure by imaging the heart as illustrated in FIG. 5a and defining within such a volumetric region a 3D ROI 96. As FIG. 5a illustrates, this 3D ROI extends into the heart chambers on both sides of the valve and includes the valve through which the catheter 100 is to be inserted. With the 3D ROI defined in this way, the 3D ROI is viewed simultaneously from both the face in the atrium 110 and the face in the ventricle as shown in FIGS. 5b and 5c. In the view V₁ from the atrium 110 as shown in FIG. 5b, the clinician can see the catheter 100′ as it approaches the slits 102 between the valve leaflets. On the other side of the valve the V2 view of FIG. 5c views the slits 102 of the valve leaflets through which the catheter will soon appear, and the chordate tendineae 104 extending back from the valve leaflets. By viewing the valve 94 from both sides in 3D, the clinician can guide the catheter 100 toward the center of the heart valve 94, and view its insertion through the heart valve as the catheter appears on the ventricular side of the valve 94.

FIGS. 6a-6c illustrate another example of a clinical procedure performed with an ultrasound system of the present invention. In this example the 3D ROI is viewed in two orthogonal viewing directions V₁ and V₂. In this example a catheter 120 is being guided to perform a clinical procedure on a spot 124 on the wall of the myocardium 90 of a heart. A 3D ROI is delineated as shown by outline 122 in FIG. 6a, which includes the catheter 120, the spot 124 which is to be treated, and the far side 126 of the heart chamber in which the procedure is to be performed. This 3D ROI 122 is viewed in two orthogonal viewing directions, V₁ as shown in FIG. 6a, and in a second direction looking into the plane of the FIG. 6a drawing. FIG. 6b illustrates the 3D ROI 122 as viewed from direction V₁. In this view the catheter 120 can be axially seen alongside the wall 90 of the myocardium and approaching the far end 126 of the heart chamber in which the catheter is located. The orthogonal V2 view is shown in FIG. 6c. In this view the catheter 120 is seen approaching point 124 at which the procedure is to be performed and is in an orientation approximately parallel to the heart wall 90. The two orthogonal views give the clinician a sense of how the catheter is proceeding along the heart wall, its spacing from the heart wall, and how much further the catheter needs to be extended to reach the point 124 at which the procedure is to be performed.

1. An ultrasonic diagnostic imaging system comprising:
an ultrasound probe operable to scan a volumetric region of a body which produces echo signals from three dimensions of the region;
a signal processor, responsive to the echo signals from the volumetric region, which produces a 3D image data set of the region;
a volume renderer coupled to receive the 3D image data set and produce two 3D views, a first 3D view and a second 3D view, of the region as if the region were being simultaneously viewed from two different viewing directions;
a first user control which selects the two different viewing directions; and
a display, responsive to the volume renderer, which simultaneously displays the two 3D views,
wherein the first and second 3D views are manipulable on the display such that moving an orientation of the first 3D view causes movement of an orientation of the second 3D view.

2. The ultrasonic diagnostic imaging system of claim 1, wherein the two different viewing directions further comprise views of the region as seen from directions oriented 180° with respect to each other.

3. The ultrasonic diagnostic imaging system of claim 1, wherein the two different viewing directions further comprise views of the region as seen from directions oriented 90° with respect to each other.

4. The ultrasonic diagnostic imaging system of claim 1, wherein the two different viewing directions further comprise views of the region as seen from directions oriented at an angle between 0° and 180° with respect to each other.

5. The ultrasonic diagnostic imaging system of claim 1, further comprising a second user control operable by a user to select a 3D region of interest (ROI) within the volumetric region, wherein the volume renderer produces two 3D views of the ROI as if the ROI were being simultaneously viewed from two different viewing directions.

6. The ultrasonic diagnostic imaging system of claim 5, further comprising an invasive object which can be seen on the display when manipulated in the volumetric region;
wherein the region of interest further contains anatomy of interest,
wherein the system is configured to visualize the invasive object can be visualized moving away from a viewer in the first 3D view one of the 3D views when manipulated in a first direction in relation to the anatomy of interest, and
wherein the system is further configured to simultaneously visualize the invasive object is simultaneously visualized moving toward the viewer in the second 3D view other of the 3D views when manipulated in the first direction in relation to the anatomy of interest.

7. The ultrasonic diagnostic imaging system of claim 5, further comprising an invasive object which can be seen on the display when manipulated in the volumetric region;
wherein the region of interest further contains anatomy of interest, wherein the system is configured to visualize the invasive object can be visualized moving toward or away from a viewer in the first 3D view one of the 3D views when manipulated in a first direction in relation to the anatomy of interest, and
wherein the system is further configured to simultaneously visualize the invasive object is simultaneously visualized moving laterally with respect to the viewer in the second 3D view other of the 3D views when manipulated in the first direction in relation to the anatomy of interest.

8. The ultrasonic diagnostic imaging system of claim 1, further comprising a third user control, coupled to the volume renderer, which is operable to change the orientation of the volumetric region as seen from the two different viewing directions.

9. The ultrasonic diagnostic imaging system of claim 8, wherein the third user control is operable to change the orientation of the volumetric region as seen from a first of the two different viewing directions,
wherein the orientation of the volumetric region as seen from the other of the two different viewing directions is changed in correspondence to the change applied to the first viewing direction,
wherein a user sees a single change in the orientation of the volumetric region as it would appear from two different viewing directions of the volumetric region.

10. The ultrasonic diagnostic imaging system of claim 9, wherein the system is configured such that the change in the orientation of the two 3D views of the volumetric region produced by manipulation of the third user control is visualized in real time.

11. The ultrasonic diagnostic imaging system of claim 8, wherein the third user control is further operable to tilt the two 3D views up or down, turn the two 3D views left or right, or rotated the two 3D views clockwise or counter-clockwise.

12. The ultrasonic diagnostic imaging system of claim 11, wherein the system is configured such that further comprising:
when the third user control is operated to tilt one of the 3D views up, the other 3D view tilts down correspondingly;
when the third user control is operated to turn one of the 3D views to the left, the other 3D view turns to the left correspondingly; and
when the third user control is operated to rotate one of the two 3D views clockwise, the other 3D view rotates counter-clockwise correspondingly.

13. The ultrasonic diagnostic imaging system of claim 1, wherein the volume renderer further comprises:
a first volume renderer which produces a 3D view of the volumetric region from a first viewing direction, and
a second volume renderer which produces a 3D view of the volumetric region from a second viewing direction.

14. The ultrasonic diagnostic imaging system of claim 13, wherein the two 3D views further comprise kinetic parallax renderings.

15. The ultrasonic diagnostic imaging system of claim 14, wherein the 3D image data set further comprises B mode or Doppler image data.
